(19) United States
(12) Patent Application Publication
(10) Pub. No.: US 2006/0147378 A1
(43) Pub. Date: Jul. 6, 2006

(54) Azulene dimer-quenched, near-infrared fluorescent probes

(76) Inventors: Ching-Hsuan Tung, Wayland, MA (US); Ralph Weissleder, Peabody, MA (US); Wellington Pham, Toledo, OH (US)

Correspondence Address: FISH & RICHARDSON PC, P.O. BOX 1022, MINNEAPOLIS, MN, 55440-1022, US

(21) Appl. No.: 10/528,634

(22) PCT Filed: Sep. 24, 2003
(86) PCT No.: PCT/US03/29610

§ 371 Date: Dec. 15, 2005

Related U.S. Application Data
(60) Provisional application No. 60/413,408, filed on Sep. 24, 2002.

(51) Int. Cl. A61K 49/00; C07K 16/46; C07K 14/47

(52) U.S. Cl. 424/9.6; 530/409; 536/18.7; 530/391.1; 564/86; 564/152

(57) ABSTRACT

An intramolecularly-quenched, near-infrared fluorescence probe that emits substantial fluorescence only after interaction with a target tissue (i.e., activation) is disclosed. The probe includes a polymeric backbone and a plurality of near-infrared fluorochromes covalently linked to the backbone at fluorescence-quenching interaction-permissive positions separable by enzymatic cleavage at fluorescence activation sites. The probe optionally includes protective chains or fluorochrome spacers, or bothours. Also disclosed are methods of using the intramolecularly-quenched, near-infrared fluorescence probes for in vivo optical imaging.

### FIELD OF THE INVENTION

The invention relates to biochemistry, cell biology, and in vivo optical imaging.

### BACKGROUND OF THE INVENTION

Fluorescence resonance-energy transfer has long been used to study various biological events in vitro, such as protease kinetics or nucleic acid hybridization. To impart high signal changes in protease assays, a fluorescent donor and a non-fluorogenic chromophore are often covalently attached to the ends of a specific enzyme substrate. Resonance-energy transfer from the excited state of a fluorophore to a non-fluorogenic chromophore results in quenching of a fluorescence signal. Upon proteolytic cleavage of the substrate by enzymes, the fluorescent dye and the quencher are separated from each other, resulting in fluorescence.

Fluorochromes typically used for the above assays fluoresce in the visible range (λ=400-650 nm), so that the fluorescence signal can be conveniently visualized by fluorescence microscopes or measured by spectrophotometers. However, light in this range is not ideal for many in vitro and in vivo applications, because of autofluorescence in the visible spectrum, and because of strong absorption of photons by tissue and blood. Recently various near-infrared (NIR, λ=700-900 nm) probes have shown promise for in vivo imaging of various target molecules or biological events, such as receptors, tumor associated proteases, osteolastic activity and thrombin. Near-infrared fluorochromes are preferable for in vivo imaging since NIR light penetrates tissue more efficiently than light in the visible spectrum. Therefore, probes, e.g., molecular beacons, in the NIR range are essential for in vivo applications. Currently there are a few commercially available NIR fluorochromes; unfortunately, high efficiency quenchers for these NIR fluorochromes have been largely lacking and this has represented a major drawback toward the development of NIR probes, e.g., molecule beacons.

### SUMMARY OF THE INVENTION

The invention is based on the discovery of new nonfluorescent bisazulene dimer quenchers, e.g., of formula (I), and their use in stable and biocompatible near-infrared fluorescence probes that contain a fluorochrome paired with the new quenchers. The azulene dimer scaffold is both a wavelength tunable and efficient absorber of near-infrared fluorescence energy. Thus, the new probes remain “optically silent” only until the above pairing is irreversibly separated via interaction with a target, e.g., a target tissue (i.e. activation). The probes of this invention are of the design that “activation” is a diagnostic cellular event. The occurrence of this event is mainfested by the readily measurable fluorescence of the liberated fluorochrome.

Accordingly, the invention features intramolecularly-quenched fluorescence probes including a bisazulene dimer quencher and a near-infrared fluorochrome, each covalently linked to an enzymatically cleavable spacer at fluorescence-quenching permissible positions. These intramolecularly-quenched probes can be further modified to include a fourth component, namely a polymeric backbone, which is attached to the probe assembly, e.g., through the spacer.

The polymeric backbone can be any biocompatible polymer. For example, it can be a polypeptide, a polysaccharide, a nucleic acid, or a synthetic polymer.

Polypeptides useful as a backbone include, for example, polylysine, albumins, and antibodies. Poly(L-lysine) is a preferred polypeptide backbone. The backbone also can be a synthetic polymer such as polyglycolic acid, polylactic acid, poly(glycolic-co-lactic) acid, polydioxanone, polyvalerolactone, poly-ε-caprolactone, poly(3-hydroxybutyrate, poly(3-hydroxyvalerate) polytartronic acid, and poly(β-malonic acid).

The probe can include one or more protective chains covalently linked to the polymeric backbone. Suitable protective chains include polyethylene glycol, methoxypolyethylene glycol, methoxypolypropylene glycol, copolymers of polyethylene glycol and methoxypolypropylene glycol, dextran, and polylactic-polyglycolic acid. In some embodiments of the invention, the backbone is polylysine and the protective chains are methoxypolyethylene glycol.

Near-infrared fluorochromes useful in this invention include Cy5.5, Cy5, Cy7, IRD41, IRD700, NIR-1, LaJolla Blue, Alexa Fluor® 680, Alexa Fluor® 450, indocyanine green (ICG) and analogs thereof, indotricarbocyanine (ITC), and chelated lanthanide compounds that display near-infrared fluorescence. The fluorochrome can be covalently linked to the spacer, using any suitable reactive group on the fluorochrome and a compatible functional group on the spacer.

The spacer can be a peptide, oligonucleotide, or other moiety, e.g., a synthetic moiety, containing degradable bonds to which near-infrared fluorochromes and quenchers are covalently linked.

A probe according to this invention also can include a targeting moiety, e.g., an antibody, antigen-binding antibody fragment, a receptor-binding polypeptide, or a receptor-binding polysaccharide. Such a moiety can be used to effect preferential accumulation of the probe in the target tissue. The targeting moiety can be incorporated into the probe via attachment to the spacer.

This invention also features new azulene dimer quenchers having a general formula (I):

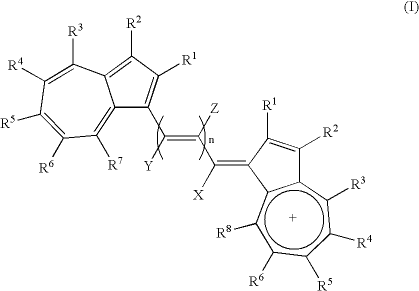

wherein:

R¹, R², R³, R⁴, R⁵ and R⁶ are independently selected from hydrogen, C₁-C₁₂ alkyl, cycloalkyl, heterocyclyl, aryl, heteroaryl, halo, hydroxy, nitro, sulfate, phosphate haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups;

n is independently 1, 2, 3 or 4;

X is independently hydrogen, carbonyl, CH═CH, or when taken together with Y may form a ring;

Y is independently hydrogen or a chemical bond when taken together with X to form a ring,

Z is independently hydrogen or an oxygen radical;

R⁷ and R⁸ are CH₂(CH₂)ₘG;

m is independently 0-12;

G is independently C(O)OH, C(O)OR⁹, C(O)O—NR¹⁰R¹¹, or O—S(O)₂R¹² with the proviso that only one G of R⁷ or R⁸ may be C(O)OR⁹;

R⁹ is C₁-C₁₂ alkyl;

R¹⁰ and R¹¹ taken together form di-oxoheterocyclyl or heteroaryl; and

R¹² is aryl optionally substituted with halo or nitro.

Embodiments may include one or more of the following features.

R⁸ can be CO₂H,

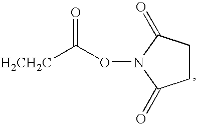

or

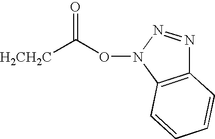

Z can be O⁻, and X and Y can combine to form squaric acid.

The invention also features new azulene dimer-quenched, near-infrared fluorescent probes having formula (I):

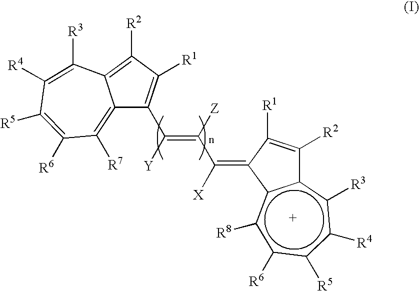

wherein:

R¹, R², R³, R⁴, R⁵, and R⁶ are independently selected from hydrogen, C₁-C₁₂ alkyl, cycloalkyl, heterocyclyl, aryl, heteroaryl, halo, hydroxy, nitro, sulfate, phosphate, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups;

n is independently 1, 2, 3 or 4;

X is independently hydrogen, carbonyl, CH═CH, or when taken together with Y may form a ring;

Y is independently hydrogen or a chemical bond when taken together with X to form a ring;

Z is independently hydrogen or an oxygen radical;

R⁷ and R⁸ are CH₂(CH₂)ₘG;

m is independently 0-12;

G is independently C(O)OR⁹, C(O)NHK, or SL with the proviso that only one G in R⁷ and R⁸ may be C(O)OR⁹;

R⁹ is C₁-C₁₂ alkyl;

K is an N-terminal amino acid of an amino acid sequence containing a fluoroscence activation site within the sequence and a near-infrared fluorochrome covalently bonded to the amino acid sequence directly or through a spacer; and

L is a cysteine residue of an amino acid sequence containing a fluoroscence activation site within the sequence and a near-infrared fluorochrome covalently bonded to the amino acid sequence directly or through a spacer.

In some embodiments, K can be glycine, the amino acid sequence can be Gly-Asp-Glu-Val-Asp-Gly-Ser-Gly-Cys-NH₂ (SEQ ID NO: 1) and the fluorochrome can be Alexa-680 C₂ maleimide.

The invention also features in vivo methods of detecting, e.g., imaging, a target, e.g., a tumor or an arthritic area in a joint, in a subject. The methods include (a) administering to a subject a probe containing an enzymatically cleavable spacer to which is covalently linked: (i) a near-infrared fluorochrome, (ii) a bisazulene dimer quencher, and optionally (iii) a polymeric backbone or targeting moiety, which accumulates preferentially in the target; (b) allowing time for (i) the probe to accumulate preferentially in the target, and (ii) enzymes in the target to activate the probe by enzymatic cleavage at a fluorescence activation site; (c) illuminating the target with near-infrared light of a wavelength absorbable by the fluorochrome; and (d) detecting fluorescence emitted by the fluorochrome. In some embodiments, (a), (b), (c), and (d) are repeated over time.

In some embodiments, the fluorescence emitted in (d) can be used to form an image of the target.

The above methods can be used, e.g., for in vivo detection, e.g., imaging, of a tumor or in vivo detection or evaluation, e.g., imaging, of arthritis in a joint. The subject may be a mammal, e.g., a human or animal.

In some embodiments, (d) can be performed using a suitable light detection or image recording component consisting of a charged coupled device (CCD) system or photographic film.

In some embodiments, the presence, absence, or level of probe activation can be indicative of a disease state, e.g., cancer.

The invention also features in vivo methods for selectively detecting two different cell or tissue types simultaneously. The methods include: (a) administering to a subject two different probes each containing an enzymatically cleavable spacer to which is covalently linked: (i) a near-infrared fluorochrome, (ii) a bisazulene dimer quencher, and optionally (iii) a polymeric backbone or targeting moiety, each of which accumulates preferentially in a target tissue, wherein each of the two probes comprises a fluorochrome whose fluorescence wavelength is distinguishable from that of the other flurorochrome, and each of the two spacers comprises a different activation site; (b) allowing time for (i) the probes to accumulate preferentially in the target tissue, and (ii) enzymes in the target tissue to activate the probes by enzymatic cleavage at a fluorescence activation site, if the target tissue is present; (c) illuminating the target tissue with near-infrared light of a wavelength absorbable by the fluorochromes; and (d) separately detecting fluorescence emitted by the two fluorochromes. The fluorescence emitted by the two different fluorochromes in (d) can be used to form an image of the two different cell or tissue types simultaneously. Other embodiments may include one or more of the above features.

As used herein, “fluorescence-quenching permissible positions” means the positions of a fluorochrome and a quencher relative to one another such that the quencher can quench the fluorochrome's fluorescence.

As used herein, “targeting moiety” means a molecule or compound bound covalently or noncovalently to a self-quenched probe, to enhance the concentration of the probe in a target tissue relative to surrounding tissue.

The term “halo” or halogen refers to any radical of fluorine, chlorine, bromine or iodine.

The term “alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. The term “haloalkyl” refers to an allyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. “Aralkyl” includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The terms “alkylamino” and “dialkylamino” refer to —NH(alkyl) and —NH(alkyl)₂ radicals respectively. The term “alkoxy” refers to an —O-alkyl radical. The 9 term aryloxy refers to an —O-aryl radical. The term “mercapto” refers to an —SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The term “cycloalkyl” as employed herein includes saturated and partially unsaturated cyclic, bicyclic, tricyclic, or polycyclic hydrocarbon groups having 3 to 12 carbons, (e.g., 3 to 8 carbons, 3 to 6 carbons), in which the cycloalkyl group additionally may be optionally substituted. Examples of cycloalkyl groups include, without limitation, cyclopropyl, cyclobutyl, cyclopentyl, cyclopentenyl, cyclohexyl, cyclohexenyl, cycloheptyl, cyclooctyl, norbornyl, and adamantyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent.

Examples of heteroaryl groups include pyridyl, furyl or furanyl, imidazolyl, benzimidazolyl, pyrimidinyl, thiophenyl or thienyl, quinolinyl, indolyl, thiazolyl, benzotriazoyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a moiety, e.g. an atom or group of bonded atoms, “substituted” on an alkyl, cycloalkyl, aryl, heterocyclyl, or heteroaryl group at any atom of that group. Suitable substituents include, without limitation, alkyl, cycloalkyl, heterocyclyl, aryl, heteroaryl, halo, hydroxy, nitro, sulfate, phosphate haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, ureido groups, and oxo. In one aspect, the substituents are independently selected from the group consisting of C₁-C₆ alkyl, C₃-C₈ cycloalkyl, (C₁-C₆)alkyl(C₃-C₈)cycloalkyl, C₂-C₈alkenyl, C₂-C₈ alkynyl, cyano, amino, C₁-C₆alkylamino, di(C₁-C₆)alkylamino, benzylamino, dibenzylamino, nitro, carboxy, carbo(C₁-C₆)alkoxy, trifluoromethyl, halogen, C₁-C₆ alkoxy, C₆-C₁₀ aryl, (C₆-C₁₀)aryl(C₁-C₆)alkyl, (C₆-C₁₀)aryl(C₁-C₆)alkoxy, hydroxy, C₁-C₆ alkylthio, C₁-C₆ alkylsulfinyl, C₁-C₆ alkylsulfonyl, C₆-C₁₀ arylthio, C₆-C₁₀ arylsulfinyl, C₆-C₁₀ arylsulfonyl, C₆-C₁₀ aryl, (C₁-C₆)alkyl(C₆-C₁₀)aryl, and halo(C₆-C₁₀)aryl.

The invention provides several advantages, particularly in the areas of cellular delivery, reduced background fluorescence, and feasibility of simultaneous multiple probe use.

Earlier generations of near-infrared fluorescent probes were based on relatively high molecular weight polymers containing relatively large numbers of fluorochromes. The plurality of the latter compounds were necessary to ensure efficient self-quenching (i.e., “silencing”) of the probe prior to activation. In the present invention, only one fluorochrome is needed (e.g., rather than dozens) since the azulene dimer molecule efficiently quenches fluoroescence. As a result, with fewer “optical” components to load, it is now possible to use much lower molecular weight materials (i.e., spacers) with markedly improved cellular delivery properties. The spacers used in this invention can contain more than one fluorochrome-quencher pair especially where improved signal amplification is desired.

The probes of this invention are activatable and do not exhibit fluorescence until interaction with the target tissue. Additionally, the quencher molecule is itself nonfluorescent and will not contribute unwanted background fluorescence once activation occurs and the fluorochrome-quencher pair is permanently disrupted.

Finally, the addition or subtraction of substituents on the azulene core enables one to generate a spectrum of quenchers which absorb at incrementally different amounts within the 650-850 nm near-infrared range. A productive end result is that multiple probes with different absorption/emission profiles can be prepared from the same molecular scaffold. The spectrally distinguishable probes can then be used simultaneously in one imaging experiment.

Unless otherwise defined, all technical and scientific terms used herein have the same meaning as commonly understood by one of ordinary skill in the art to which this invention belongs. In case of conflict, the present application, including definitions will control. All publications, patent applications, patents, and other references mentioned herein are incorporated by reference in their entirety.

Although methods and materials similar or equivalent to those described herein can be used in the practice of the present invention, preferred methods and materials are described below. The materials, methods, and examples are illustrative only and not intended to be limiting. Other features and advantages of the invention will be apparent from the detailed description and from the claims.

### BRIEF DESCRIPTION OF THE DRAWINGS

FIG. 1 is a graph of the absorption spectra for an azulene monomer and three azulene dimers derived from squaric acid.

FIG. 2 is a graph of the absorption spectra for NIRQ750 and the dye Alexa Fluor® 680 C₂ maleimide.

FIG. 3 is a schematic of a self-quenched fluorescence probe and its activation.

FIG. 4 is a flow diagram showing the assembly of NIR-fluorescent caspase −3 substrates.

FIG. 5 is a series of representations of chemical structures of Cy5.5, CY5, IRD41, and IRD 700.

FIG. 6 is a series of representations of chemical structures of NIR 1 and LaJolla Blue.

FIG. 7A is a representation of probes 6, 7, and 8. FIG.

FIG. 7B is a graphical representation of fluorescence with and without caspase-3 inhibitor.

FIG. 7C is a bar graph showing changes in the intensity of the NIR fluorescence signal (n-fold) at 180 minutes for five activation experiments.

### DETAILED DESCRIPTION

Quenchers

The new quenchers are dimers in which two azulene molecules are linked through their respective five-membered rings by a pi-conjugating linker. The pi system of the linker further extends the delocalized azulene pi system and results in new chromophores, which exhibits an absorbance maximum in the near-infrared portion of the spectrum. The red-shifting of the dimer λₘₐₓ (about 700-800 nm) is significant with respect to that of the monomeric substrate (about 500-600 nm). Minimally substituted azulenes, in fact, result in dimers with the preferred absorption characteristics.

The quencher is covalently attached to the spacer during probe assembly and use. Therefore it is essential that an electrophilic group, e.g., an amine or sulfur reactive group, be present for bioconjugation.

The new quenchers are generally described by the general formula (1). As noted, azulenes having minimal substitution produce dimers with the desired absorbance properties. Substituents R¹-R⁶ may be varied either to “tune” further the scaffold to a desired wavelenth, or where necessary, increase the hydrophilic nature of the quencher. The linker portion of the quencher may also be used to tune absorbance wavelength and may either be a linear or cyclic moiety. For the embodiments of the present invention, the electrophilic group for bioconjugation is located on R⁷ or R⁸.

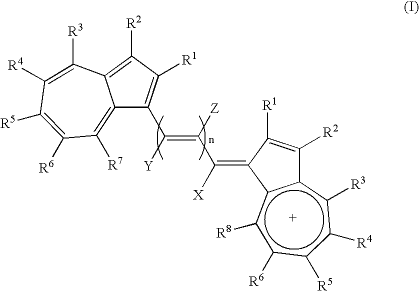

It is understood that the actual electronic structure of some chemical entities cannot be adequately represented by only one canonical form (e.g., a Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art generally recognizes as the predominant resonance form for a particular species.

Embodiments of the present invention include as structures (II) and (III) where 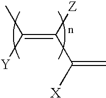
in formula (I)
is derived from squaric acid. These quenchers contain carboxylic acid groups which may be readily converted to activated esters for subsequent bioconjugation to the spacer. Examples of activated esters are those derived from N-hydroxysuccinimide or 1-hydroxybenzotriazole. A comparison of the absorption data for Quencher NIRQ750 (II) (λmax 760) and NIRQ700 (III) (λmax 700) reveals that the presence of the isopropyl group on the former redshifts the λmax by 60 nm. This observation demonstrates that quenchers with discretely different absorption properties can be realized by relatively straightforward modifications in the azulene core substitution pattern.

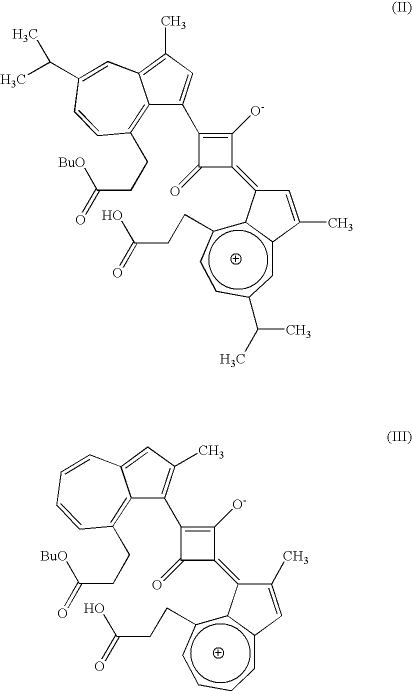

In general, the quenchers may be obtained upon dimerization of an appropriately substituted azulene in the presence of a second component which ultimately becomes the pi-conjugating linker (see Scheme 1 below). It is preferable to have the bioconjugation “handle” present on the azulene prior to the dimerization. If not already present, this “handle” may be incorporated by first introducing a methyl group into the 4-position of the azulene to form (IV). Deprotonation followed by addition of an electrophile bearing groups, e.g., a carboxy group, produces (V) containing the critical bioconjugation handle (see Scheme 2 below). Azulene monomers may be obtained commercially or synthesized from monocyclic precursors, e.g., (VI).

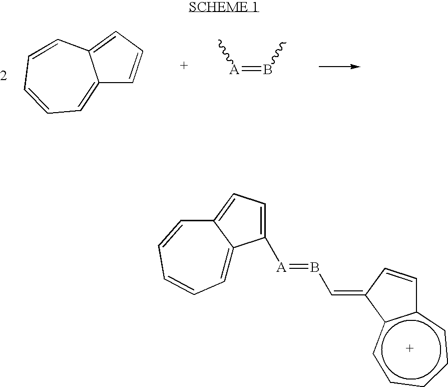

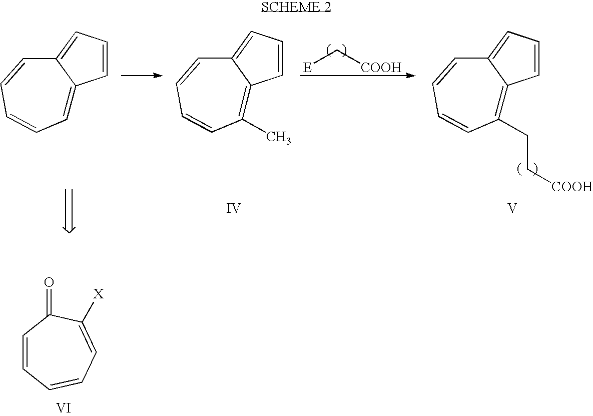

The synthesis of NIRQ₇₀₀ (III) (see Scheme 3 below) is started with a known procedure (Yokota, T.; Chem. Pharm. Bull., 1994, 865) by reacting tropolone 1 activated in situ as tropolone tosylate with dimethyl malonate in the presence of sodium methoxide to obtain lactone 2. The crude product is recrystallized using ethanol and dichloromethane to provide 2 in 84% yield. The azulene ring is prepared via [8+2] cycloaddition of lactone 2 with vinyl ether, which is a product of thermolysis of acetals (Nozoe, T., et al. Heterocycles 1990, 31, 17; Pham, W., et al. Tetrahedron Letters 2001, 43, 19). This reaction is temperature and solvent dependent. In the absence of solvent, only a black tar-like decomposition material is recovered. The expected brownish-red liquid product 3 is obtained by heating 2 with 2,2-dimethoxypropane in anhydrous toluene at 200° C. under pressure (Pham, W., et al. Tetrahedron Letters 2001, 43, 19).

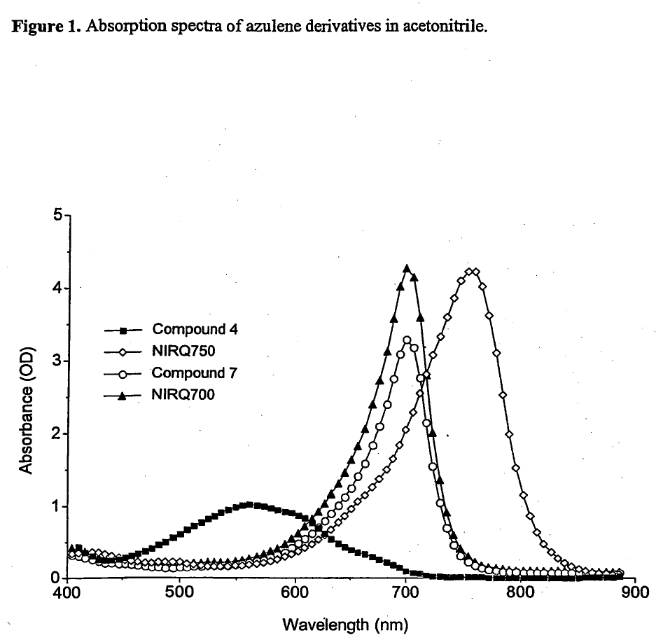

The ester of 3 is removed by saponification using 2M KOH and the product is recrystallized with methanol and chloroform to yield the pink solid acid 4. Subsequent condensation is carried out by refluxing compound 4 with squaric acid; unfortunately, instead of obtaining the expected azulenyl squaraine product 5 with the intact free carboxylic acid, an unexpected product 7 is observed. The absorption of 7 (green color) is found to be about 180 nm higher than compound 4 in acetonitrile clearly demonstrating that the condensation process was successful (FIG. 1). The structural assignment for 7 is supported by ¹H NMR. When compound 3 is treated with anhydrous phosphoric acid (deliberate decarboxylation conditions), conversion to 7 is complete in 5 minutes at 100° C. During the reaction, the color changes from brownish red (3) to purple (6).

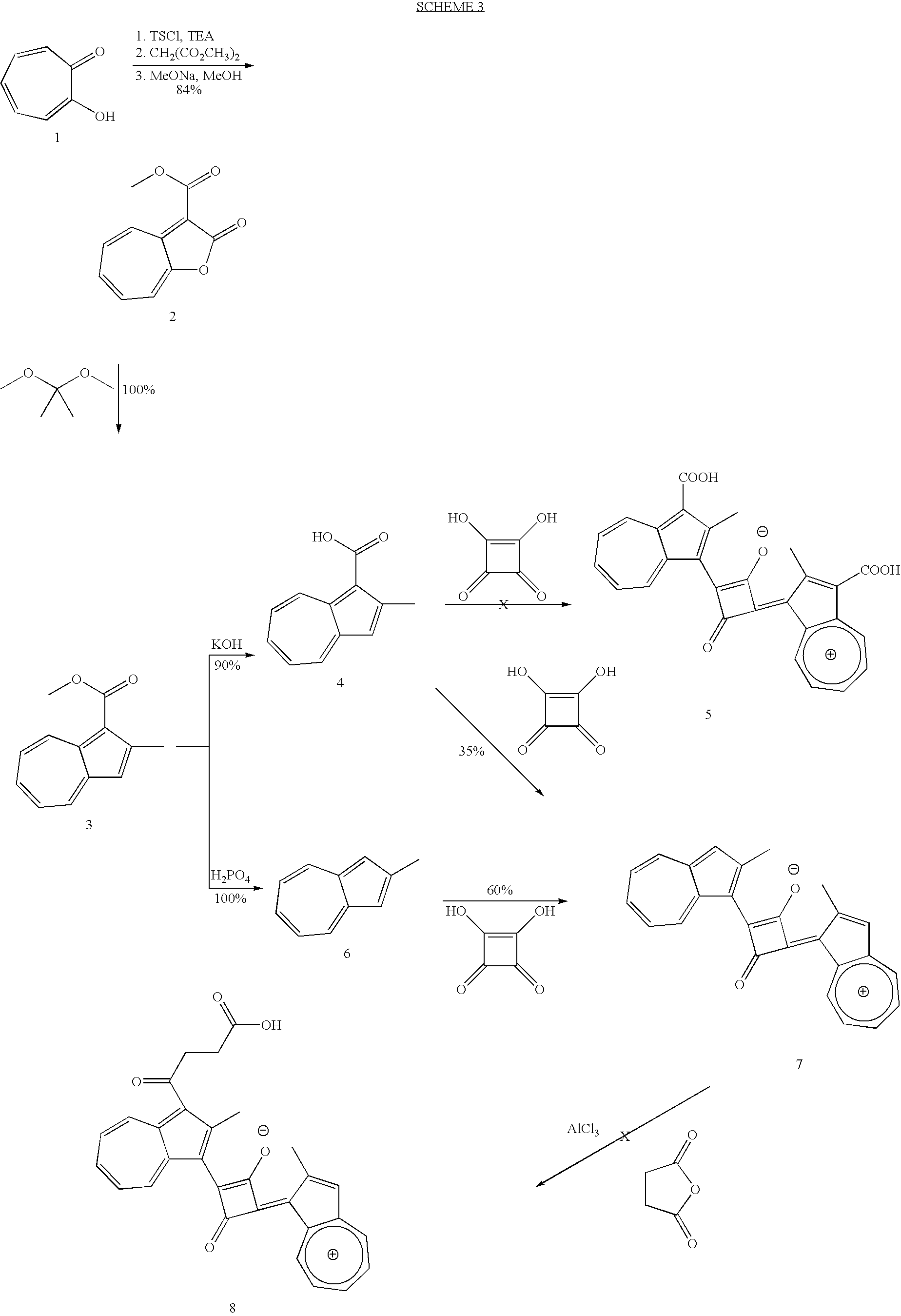

An alternative approach to preparing monofunctional squaraine azulene analogs is started from methylation on the 7-membered ring of compound 6 with methyllithium at room temperature in diethyl ether (see Scheme 4 below). The mixture is refluxed overnight to form the azulenate ions of the Meisenheimer-type intermediate (Hafner, K., et al. Tetrahedron 1992, 48, 4879; McDonald, R N., et al. J. Org. Chem. 1974, 39, 1877) as the color changes from deep blue to a pale yellow suspension. Addition of methanol at −70° C. provides a colorless solution which in turn is treated with p-chloranil to give 2,4-dimethyl azulene 9 as a dark blue oil in 52% yield efficiency (Chen, S. L.; Klein, P; Hafner, K. Eur. J. Org. Chem. 1998, 423).

The absorbance maximum of 2,4-dimethyl azulene 9 is similar to intermediate 4. A carboxylic moiety was then introduced by nucleophilic substitution with bromoacetic acid (Schrott, W., et al. Eur. Pat. Appl., EP 310080, 1989). After mixing 2,4-dimethyl azulene 9 with n-BuLi at −40° C. in the presence of diisopropyl amine, the nucleophilic substitution is carried out by adding bromoacetic acid dropwise to the reaction mixture. At the end of the reaction the suspension is acidified with 2M HCl and the carboxylic product 10 is extracted with diethyl ether. Condensation between 10 and squaric acid in refluxing n-butanol provided the mono- and di-ester, NIRQ₇₀₀ and 11 in 26% and 38% yield, respectively. The prepared NIRQ₇₀₀ has an absoption maximum at λₘₐₓ=700 nm and a broad spectrum between λ=600 and 750 nm (FIG. 1).

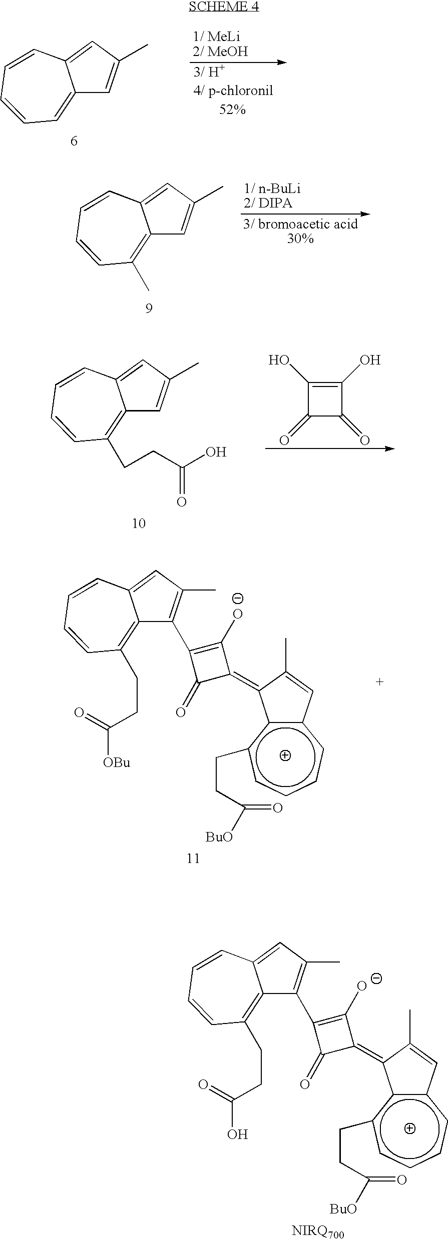

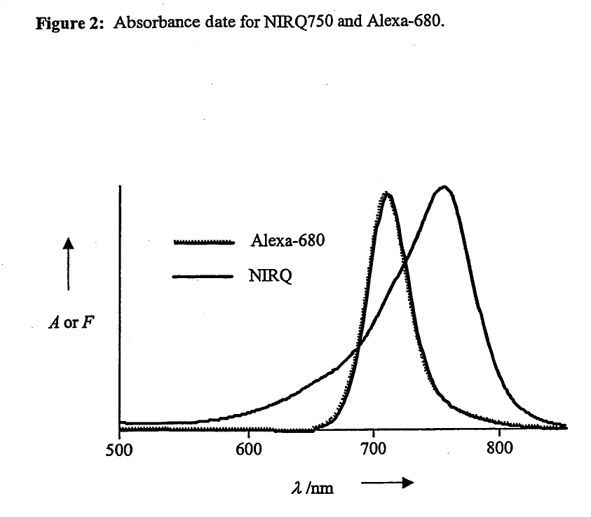

The synthesis of non-fluorogenic near-infrared quencher NIRQ750 (III) begins with commercially available guaiazulene 12 (see Scheme 5 below). A carboxylic moiety is first introduced into the cycloheptatriene ring as a handle prior to condensation. Deprotonation of the methyl group at the 4-position is performed using Schrott's reaction (Schrott, W., et al. Eur. Pat. Appl. EP 310080, 1989). The nucleophilic moiety is generated by n-BuLi at −40° C. in the presence of diisopropylamine. The subsequent in situ nucleophilic substitution is carried out by reaction with bromoacetic acid to make the carboxylic intermediary 13. Two moles of the carboxylic analog 13 are then condensed with a mole of squaric acid by refluxing in the presence of n-BuOH. The dominant product of this reaction is the desired mono-substituted dimer NIRQ750 (III). Under optimized conditions, a di-substituted by-product is maintained at less than 4% and is removed conveniently by flash column chromatography. As expected, a significant absorption shift from 550 nm to a broad peak around 700-800 nm (FIGS. 1 and 2) was observed upon dimerization. The absorption maximum (δmax) changed little with different solvents; however, the extinction coefficient (ε) is solvent dependent (Table 1 below).

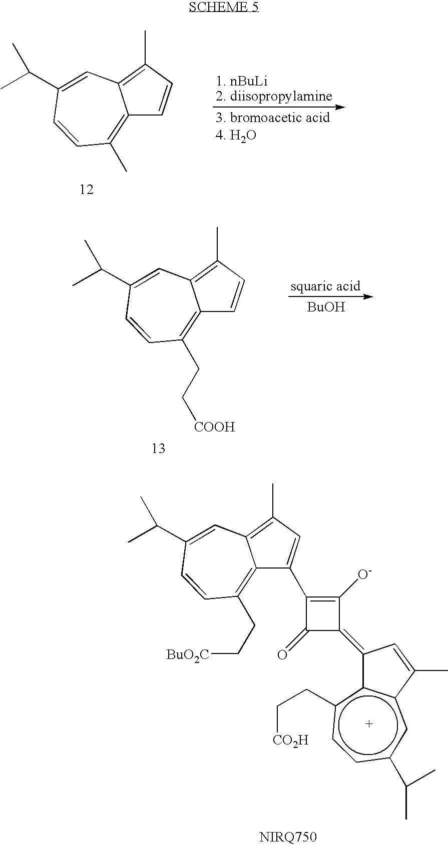

TABLE 1
Absorption maximum and extinction coefficient of NIRQ.

| Solvent | δmax (nm) | ε (M⁻¹ cm⁻¹) |
|---|---|---|
| Acetonitrile | 760 | 69,000 |
| Methanol | 757 | 82,000 |
| Dimethylformamide (DMF) | 768 | 50,000 |
| Dimethylsulfoxide (DMSO) | 773 | 41,000 |

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps can be performed in an alternate sequence or order to give the desired compounds. Synthetic chemistry transformations and protecting group methodologies (protection and deprotection) useful in synthesizing the compounds described herein are known in the art and include, for example, those such as described in, e.g., R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The new compounds may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in allylation at multiple sites, the invention expressly includes all such reaction products). All such isomeric forms of such compounds are expressly included in the present invention. All crystal forms of the compounds described herein are expressly included in the present invention.

Probe Design and Synthesis

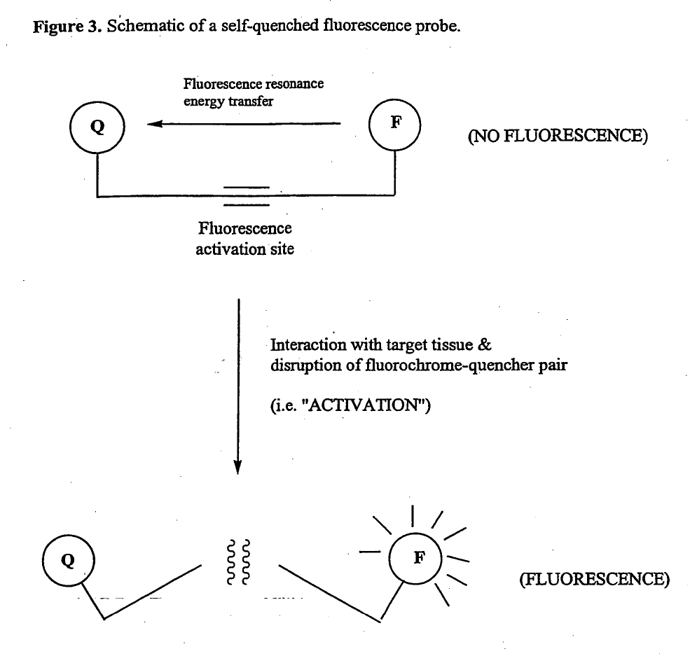

A schematic depiction of an intramolecularly-quenched fluorescence probe is shown in FIG. 3. The fluorochrome and quencher are covalently linked to a spacer which may be a peptide, oligonucleotide or other moiety, e.g., a synthetic moiety, containing degradable bonds, e.g., amide or phosphodiester bonds. For the probe to detect a diagnostic cellular event, the spacer must contain a fluorescence activation site as part of its sequence, e.g., amino acid or nucleotide. The spacer may optionally be attached to a polymeric backbone or targeting moiety. Fluorescence resonance energy transfer from the fluorochrome to the quencher renders the probe optically silent. Upon interaction with a target at the fluorescence activation site, i.e., activation, the fluorochrome-quencher pair is irreversibly disrupted. At this point, detectable and quantifiable fluorescence occurs.

Probe architecture can include a spacer with a fluorochrome at one end and a quencher at the other end. Alternatively, the fluorochrome and quencher can be located at positions other than the terminal ends, or more than one fluorochrome-quencher pair can be present on the spacer, as long as they are located at fluorescence-quenching permissible positions.

For NIR enzyme sensing probes, an enzyme-specific amino acid sequence can occur at a point along the peptide spacer between the two optical components. Upon interaction with an enzyme (i.e. activation), the spacer is cleaved, separating the fluorochrome and the quencher. When the fluorochrome is liberated, fluorescence is observed.

NIR molecular beacons contain a fluorochrome and a quencher, which are attached to an oligonucleotide spacer and held in a quenching permissible conformation (optically silent state). The spacer contains a nucleic acid molecule-specific sequence and, upon interaction with the target (i.e. activation), hybridization of the probe occurs. This event imposes a drastic conformational change that prevents fluorescence resonance energy transfer between the two optical components. Thus, the resulting target-probe hybrid is fluorescent and detectable.

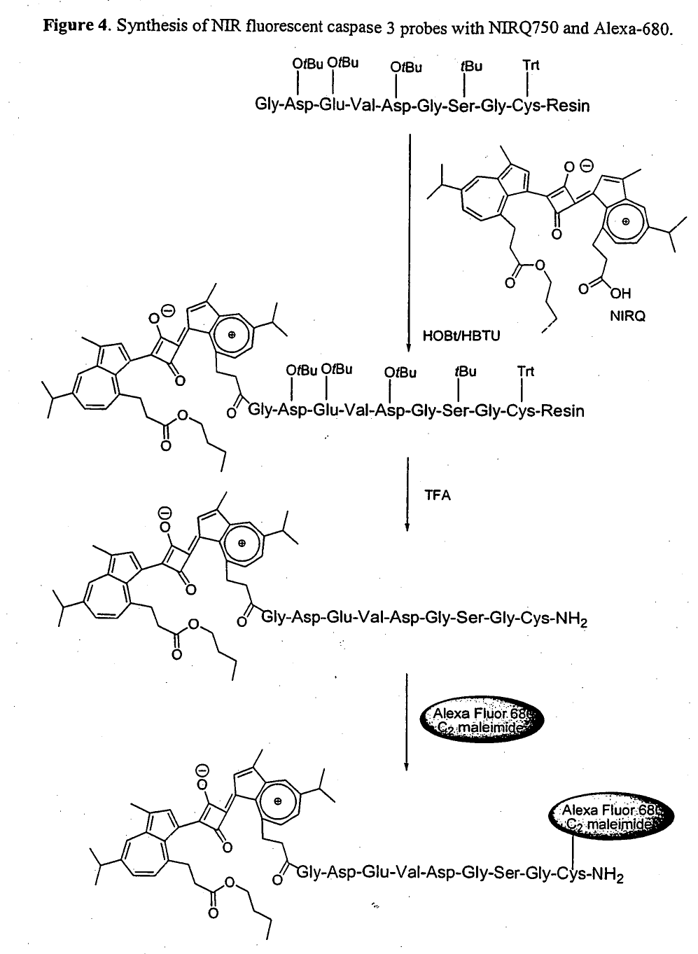

The new probes can be assembled as shown in FIG. 4. The carboxylic acid moiety of the quencher can be activated (e.g., with HOBt/HBTU) and then coupled to the resin bound peptide spacer through the N-terminal glycine residue. The quencher-containing resin can be coupled with the fluorochrome in analogous fashion after separation from the resin and global deprotection of the amino acid residues.

In some embodiments, the probe includes a peptide spacer containing only a relatively small number of amino acids, e.g., 5 to 20 amino acids, with a fluorochrome and a quencher attached to amino acids on opposite sides of a protease cleavage (i.e., activation) site. Guidance concerning various probe components, including polymeric backbone, protective side chains, fluorochromes, fluorochrome attachment moieties, spacers, fluorescence activation sites, and targeting moieties is provided in the paragraphs below. See also, Weissleder et al., U.S. Pat. No. 6,083,486.

Probe polymeric backbone design will depend on considerations such as biocompatibility (e.g., toxicity and immunogenicity), serum half-life, useful functional groups (e.g., for conjugating spacers, and protective groups), and cost. Useful types of polymeric backbones include polypeptides (polyamino acids), polyethyleneamines, polysaccharides, aminated polysaccharides, aminated oligosaccharides, polyamidoamines, polyacrylic acids and polyalcohols. In some embodiments the backbone includes a polypeptide formed from L-amino acids, D-amino acids, or a combination thereof. Such a polypeptide can be, e.g., a polypeptide identical or similar to a naturally occurring protein such as albumin, a homopolymer such as polylysine, or a copolymer such as a D-tyr-D-lys copolymer. When lysine residues are present in the polymeric backbone, the e-amino groups on the side chains of the lysine residues can serve as convenient reactive groups for covalent linkage to the spacers. When the polymeric backbone is a polypeptide, preferably the molecular weight of the probe is from 2 kD to 1000 kD. More preferably, its molecular weight is from 4 kd to 500 kd.

A polymeric backbone may be chosen or designed so as to have a suitably long in vivo persistence (half-life) inherently. Therefore, protective chains are not necessary in some embodiments of the invention. Alternatively, a rapidly-biodegradable polymeric backbone such as polylysine can be used in combination with covalently-linked protective chains. Examples of useful protective chains include polyethylene glycol (PEG), methoxypolyethylene glycol (MPEG), methoxypolypropylene glycol, polyethylene glycol-diacid, polyethylene glycol monoamine, MPEG monoamine, MPEG hydrazide, and MPEG imidazole. The protective chain can also be a block-copolymer of PEG and a different polymer such as a polypeptide, polysaccharide, polyamidoamine, polyethyleneamine or polynucleotide. Synthetic, biocompatible polymers are discussed generally in Holland et al., 1992, “Biodegradable Polymers,” Advances in Pharmaceutical Sciences, 6:101-164.

A useful polymeric backbone-protective chain combination is methoxypoly(ethylene)glycol-succinyl-N-ε-poly-L-lysyine (PL-MPEG). The synthesis of this material, and other polylysine backbones with protective chains, is described in Bogdanov et al., U.S. Pat. No. 5,593,658 and Bogdanov et al., 1995, Advanced Drug Delivery Reviews, 16:335-348.

Various near-infrared fluorochromes are commercially available and can be used to construct probes according to this invention. Exemplary fluorochromes include the following: Cy5.5, Cy5 and Cy7 (Amersham, Arlington Hts., IL; IRD41 and IRD700 (LI-COR, Lincoln, Nebr.); Alexa Fluor® 680, Alexa Fluor® 450 (Molecular Probes); NIR-1, (Dejindo, Kumamoto, Japan); LaJolla Blue (Diatron, Miami, Fla.); indocyanine green (ICG) and its analogs (Licha et al., 1996, SPIE 2927:192-198; Ito et al., U.S. Pat. No. 5,968,479); indotricarbocyanine (ITC; WO 98/47538), and chelated lanthanide compounds. Fluorescent lanthanide metals include europium and terbium.

Fluorescence properties of lanthanides are described in Lackowicz, 1999, Principles of Fluorescence Spectroscopy, 2^{nd} Ed., Kluwar Academic, New York.

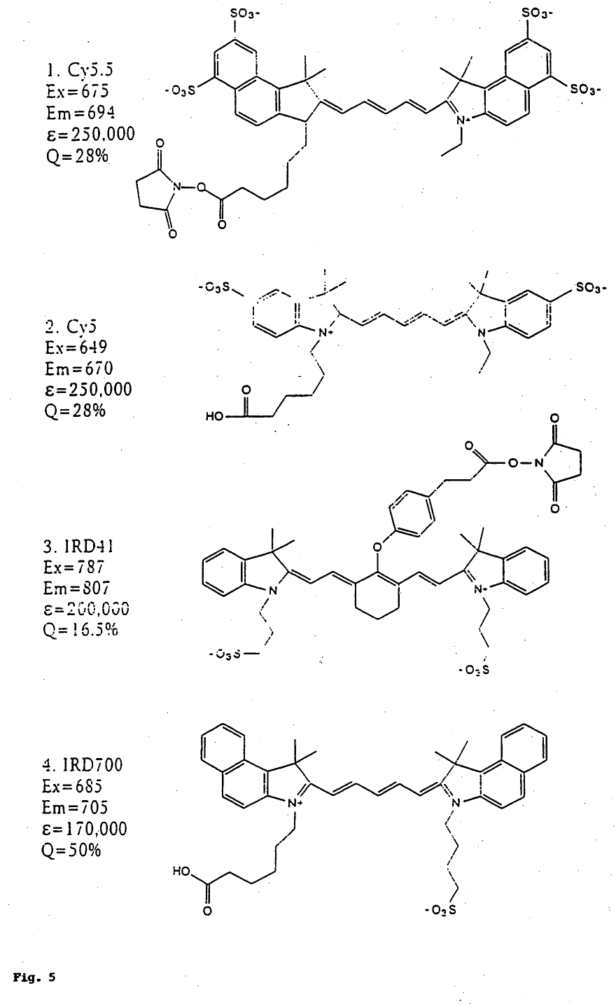

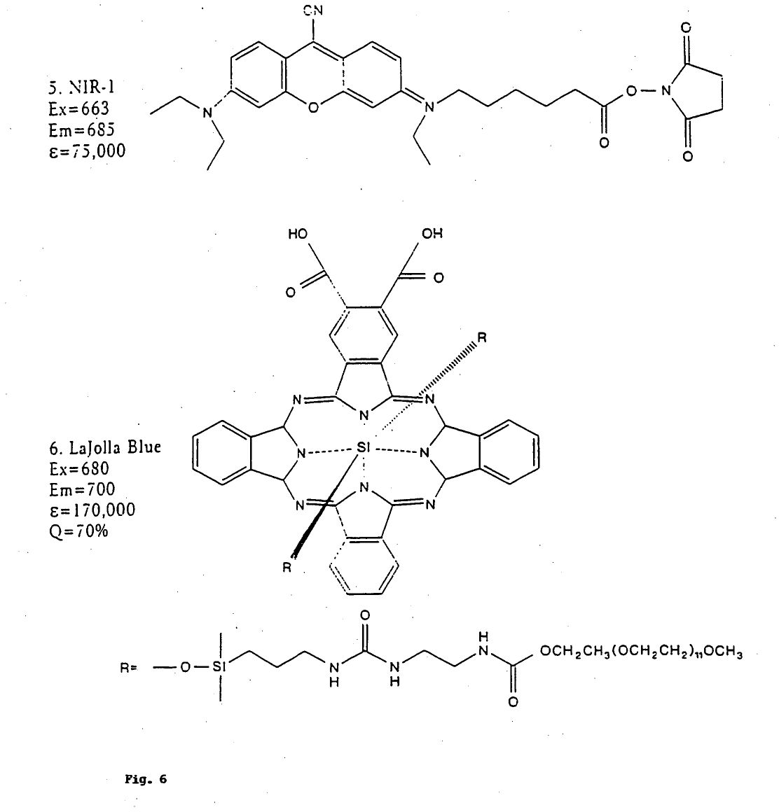

Fluorescent probes with excitation and emission wavelengths in the near-infrared spectrum are used, i.e., 650-1300 nm. Use of this portion of the electromagnetic spectrum maximizes tissue penetration and minimizes absorption by physiologically abundant absorbers such as hemoglobin (<650 nm) and water (>1200 nm). Ideal near-infrared fluorochromes for in vivo use exhibit: (1) narrow spectral characteristics, (2) high sensitivity (quantum yield), (3) biocompatibility, and (4) decoupled absorption and excitation spectra. Table 2 summarizes information on the properties of six commercially-available near-infrared fluorochromes, whose structures are shown in FIGS. 5 and 6.

TABLE 2
Exemplary Near-infrared Fluorochromes

| Fluoro- | λ(nm) | λ(nm) | | Extinct. | Quantum |
|---|---|---|---|---|---|
| chrome | excitation | emission | Mol. Wt. | Coef. | yield % |
| Cy5.5 | 675 | 694 | 1128.41 | 250,000 | 28.0 |
| Cy5 | 649 | 670 | 791.99 | 250,000 | 28.0 |
| IRD41 | 787 | 807 | 925.10 | 200,000 | 16.5 |
| IRD700 | 685 | 705 | 704.92 | 170,000 | 50.0 |
| NIR-1 | 663 | 685 | 567.08 | 75,000 | NA |
| LaJolla | 680 | 700 | 5000.00 | 170,000 | 70.0 |
| Blue | | | | | |
| Cy7 | 743 | 767 | 818.02 | 200,000 | 28.0 |
| ICG | 780 | 812 | 774.98 | 115,000 | 1.2 |
| ITC* | | | | | |

*See WO 98/47538

The fluorochrome can be covalently linked to the spacer using any suitable reactive group on the fluorochrome and a compatible functional group on the spacer. For example, a carboxyl group (or activated ester) on a fluorochrome can be used to form an amide linkage with the amino group of a peptide N-terminal amino acid. Alternatively, a fluorochrome attachment moiety can be used to tether the fluorochrome to the spacer if desired.

In all embodiments of the invention, a fluorochrome and a quencher in fluorescence-quenching positions are linked to a spacer containing an activation site between the two optical components, and this activatable fluorescence module can be used as a probe per se. In some embodiments, however, the spacer may be covalently attached to a polymeric backbone (carrier) or targeting moiety, e.g., an albumin, antibody, receptor binding molecule, synthetic polymer or polysaccharide. A useful conjugation strategy is to place a cysteine residue at the N-terminus or C-terminus of the spacer and then employ SPDP for covalent linkage between the side chain of the terminal cysteine residue and a free amino group of the carrier or targeting molecule.

Prostate Specific Antigen (PSA), is a 33 kD chymotrypsin-like serine protease is secreted exclusively by prostatic epithelial cells. Normally, this enzyme is primarily involved in post-ejaculation degradation of the major human seminal protein. Normally, serum concentrations of PSA are proportional to the volume of prostatic epithelium. The release of PSA from prostate tumor cells, however, is about 30-fold higher than that from normal prostate epithelium cells. Damaged basal membrane and deranged tissue architecture allow PSA to be secreted directly into the extracellular space and into the blood. Although high levels of PSA can be detected in serum, the serum PSA exists as a complex with a1-antichymotrypsin protein, and is proteolytically inactive. Free, uncomplexed, activated PSA occurs in the extracellular fluid from malignant prostate tissues, and PSA activity can be used as a marker for prostate tumor tissue. Prostate tumor tissue is highly enriched in PSA. Thus, spacers containing the amino acid sequence recognized by PSA can be used to produce a near-infrared probe that undergoes fluorescence activation specifically in prostate tumor tissue. PSA-sensitive spacers can be designed using information known in the art regarding the substrate specificity of PSA. See, e.g., 1997, Denmeade et al., Cancer Res. 57:4924-4930.

Cathepsin D is an abundant lysosomal aspartic protease distributed in various mammalian tissues. In most breast cancer tumors, cathepsin D is found at levels from 2-fold to 50-fold greater than levels found in fibroblasts or normal mammary gland cells. Thus, cathepsin D can be a useful marker for breast cancer. Spacers containing the amino acid sequence recognized by cathepsin D can be used to produce a near-infrared probe that undergoes fluorescence activation specifically in breast cancer tissue. Cathepsin D-sensitive spacers can be designed using information known in the art regarding the substrate specificity of cathepsin D. See, e.g., Gulnik et al., 1997, FEBS Let. 413:379-384.

Various other enzymes can be exploited to provide probe activation (cleavage) in particular target tissues in particular diseases. Table 3 provides information on 5 exemplary enzymes (including substrate sequence recognized and cleavage point) and associated diseases.

TABLE 3
Enzyme-Disease Associations

| Enzyme | Disease | Substrate | Reference |
|---|---|---|---|
| Cathepsin B/H | Cancer | K^{•}K | |
| Cathepsin D | Breast ca > | PIC(Et)F^{•}F | Gulnik, 1997, |
| | others | | FEBS Lett. |
| | | | 413: 379. |
| PSA | Prostate ca | HSSKLQ^{•} | Denmeade, |
| | | | 1997, Cancer |
| | | | Res. 57: 4924. |
| MMP's | Metastases, | P(L/Q)G^{•}(I/L)AG | Verheijen, |
| | inflammation | | 1997, Biochem. |
| | | | J. 323: 603. |
| CMV protease | Viral | GVVQA^{•}SCRLA | Sardana, 1994, |
| | | | J. Biol. Chem. |
| | | | 269: 14337 |

*Bullet (^{•}) indicates cleavage point.

Preferential accumulation of a probe in a target tissue can be achieved or enhanced by binding a tissue-specific targeting moiety (targeting ligand) to the probe through the spacer. The binding can be covalent or non-covalent. Examples of targeting moieties include a monoclonal antibody (or antigen-binding antibody fragment) directed against a target-specific marker, a receptor-binding polypeptide directed to a target-specific receptor, and a receptor-binding polysaccharide directed against a target-specific receptor. Antibodies or antibody fragments can be produced and conjugated to probes of this invention using conventional antibody technology (see, e.g., Folli et al., 1994, “Antibody-Indocyanin Conjugates for Immunophotodetection of Human Squamous Cell Carcinoma in Nude Mice,” Cancer Res. 54:2643-2649; Neri et al., 1997, “Targeting By Affinity-Matured Recombinant Antibody Fragments of an Angiogenesis Associated Fibronectin Isoform,” Nature Biotechnology 15:1271-1275). Similarly, receptor-binding polypeptides and receptor-binding polysaccharides can be produced and conjugated to probes of this invention using known techniques.

In Vitro Probe Testing

After a probe is designed and synthesized, it can be tested routinely in vitro to verify a requisite level of intramolecular fluorescence quenching before activation. Preferably, this is done by obtaining a fluorescence value for the intramolecularly quenched probe in a dilute, physiological buffer. This value is then compared to the fluorescence value obtained from an equimolar concentration of free fluorochrome in the same buffer, under the same fluorescence-measuring conditions. Preferably, this comparison will be done at a series of dilutions, to verify that the measurements are taking place on a linear portion of the fluorescence vs. fluorochrome concentration curve.

The molar amount of an intramolecularly-quenched fluorochrome on a probe can be determined by one of ordinary skill in the art using any suitable technique. For example, the molar amount can be determined readily by near-infrared absorption measurements. Alternatively, it can be determined readily by measuring the loss of reactive linking groups on the spacers, e.g., decrease in ninhydrin reactivity due to loss of amino groups.

After suitable intramolecular fluorescence quenching is verified, “de-quenching,” i.e., fluorescence, upon exposure to an activating enzyme also can be verified in vitro. In a preferred procedure, fluorescence of an intramolecularly-quenched probe is measured before and after treatment with an activating enzyme.

In addition, cells grown in culture can be used routinely to test intramolecularly-quenched near-infrared fluorescence probes. Probe molecules free in cell culture medium should be non-detectable by fluorescence microscopy. Cellular uptake should result in probe activation and a fluorescence signal from probe-containing cells. Microscopy of cultured cells thus can be used to verify that activation takes place upon cellular uptake of a probe being tested. Microscopy of cells in culture is also a convenient means for determining whether activation occurs in one or more subcellular compartments.

In Vivo Near-Infrared Imaging

Although the invention involves novel near-infrared fluorescence probes, general principles of fluorescence, optical image acquisition, and image processing can be applied in the practice of the invention. For a review of optical imaging techniques, see, e.g., Alfano et al., 1997, “Advances in Optical Imaging of Biomedical Media,” Ann. NY Acad. Sci 820:248-270.

An imaging system useful in the practice of this invention typically includes three basic components: (1) a near-infrared light source, (2) a means for separating or distinguishing fluorescence emissions from light used for fluorochrome excitation, and (3) a detection system.

The light source provides monochromatic (or substantially monochromatic) near-infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., 1994, Proc. Natl. Acad. Sci. USA 91:4887-4891; Ntziachristos et al., 2000, Proc. Natl. Acad. Sci. USA 97:2767-2772; Alexander, 1991, J. Clin. Laser Med. Surg. 9:416-418. Information on near-infrared lasers for imaging can be found on the internet at: imds.com.

A high pass filter (700 nm) can be used to separate fluorescence emissions from excitation light. A suitable high pass filter is commercially available from Omega Optical (Brattleboro, Vt.).

In general, the light detection system includes a light gathering/image forming component and a light detection/image recording component. Although the light detection system may be a single integrated device that incorporates both components, the light gathering/image forming component and light detection/image recording component will be discussed separately.

A particularly useful light gathering/image forming component is an endoscope. Endoscopic devices and techniques which have been used for in vivo optical imaging of numerous tissues and organs, including peritoneum (Gahlen et al., 1999, J. Photochem. Photobiol. B 52:131-135), ovarian cancer (Major et al., 1997, Gynecol. Oncol. 66:122-132), colon (Mycek et al., 1998, Gastrointest. Endosc. 48:390-394; Stepp et al., 1998, Endoscopy 30:379-386) bile ducts (Izuishi et al., 1999, Hepatogastroenterology 46:804-807), stomach (Abe et al., 2000, Endoscopy 32:281-286), bladder Kriegmair et al., 1999, Urol. Int. 63:27-31; Riedl et al., 1999, J. Endourol. 13:755-759), and brain (Ward, 1998, J. Laser Appl. 10:224-228) can be employed in the practice of the present invention.

Other types of light gathering components useful in the invention are catheter-based devices, including fiber optics devices. Such devices are particularly suitable for intravascular imaging. See, e.g., Tearney et al., 1997, Science 276:2037-2039; Proc. Natl. Acad. Sci. USA 94:4256-4261.

Still other imaging technologies, including phased array technology (Boas et al., 1994, Proc. Natl. Acad. Sci. USA 91:4887-4891; Chance, 1998, Ann. NY Acad. Sci. 838:2945), diffuse optical tomography (Cheng et al., 1998, Optics Express 3:118-123; Siegel et al., 1999, Optics Express 4:287-298), intravital microscopy (Dellian et al., 2000, Br. J. Cancer 82:1513-1518; Monsky et al, 1999, Cancer Res. 59:4129-4135; Fukumura et al., 1998, Cell 94:715-725), and confocal imaging (Korlach et al., 1999, Proc. Natl. Acad. Sci. USA 96:8461-8466; Rajadhyaksha et al., 1995, J. Invest. Dermatol. 104:946-952; Gonzalez et al., 1999, J. Med. 30:337-356) can be employed in the practice of the present invention.

Any suitable light detection/image recording component, e.g., a charge coupled device (CCD) system or photographic film, can be used in the invention. The choice of light detection/image recording will depend on factors including type of light gathering/image forming component being used. Selecting suitable components, assembling them into a near-infrared imaging system, and operating the system is within ordinary skill in the art.

In some embodiments of the invention, two (or more) probes containing: (1) fluorochromes that emit fluorescence at different near-infrared wavelengths, and (2) activation sites recognized by different enzymes, e.g., cathepsin D and MMP2, are used simultaneously. This allows simultaneous evaluation of two (or more) biological phenomena

### EXAMPLES

In order that the invention may be more fully understood, the following examples are provided. It should be understood that these examples are for illustrative purposes only and are not to be construed as limiting the invention in any way.

Quencher Synthesis

### Example 1

### Preparation of Tropolone tosylate

Triethylamine (114.30 μl, 0.82 mmol) was added dropwise to a round, dried flask containing a colorless solution of tropolone 1 (100 mg, 0.82 mmol) and tosyl chloride (171.90 mg, 0.90 mmol) in CH₂Cl₂ (5 mL) at room temperature and stirred for 3 hours. The reaction was poured onto ice-cold water (20 mL), and the crude product was extracted with CH₂Cl₂, dried over MgSO₄, filtered, and concentrated by rotavapor. The crude product was recrystallized from 8:2 CH₂Cl₂/hexanes to afford 190 mg (84%) of a hygroscopic brown solid: R_{f}=0.25 (9.5:0.2 CH₂Cl₂/MeOH); ¹H NMR (400 MHz, CDCl₃) δ 2.45 (s, 3H), 6.95-7.23 (m, 4H), 7.36 (d, J=8.0 Hz, 2H), 7.45 (d, J=8.5 Hz, 1H), 7.92 (d, J=6.7 Hz, 2H); ¹³C NMR (400 MHz, CDCl₃) δ 21.8, 128.6, 129.6, 130.0, 131.0, 134.6, 136.3, 141.2, 145.0; LRMS (FAB⁺) calcd (M+H)⁺ (C₁₄H₁₂O₄S) 277.31, found 277.13

### Example 2

### 2H-3-methoxycarbonylcyclohepta[b]furan-2-one (2)

A solution of NaOMe (67 mg, 1.24 mmol) in anhydrous MeOH (5 ml) was canulated to a dried flask containing a clear solution of tropolone tosylate (170 mg, 0.62 mmol) and dimethyl malonate (141.70 μl, 1.24 mmol) in MeOH (20 mL) at 0° C. The reaction turned yellow at the end of the addition. The reaction mixture was allowed to warm slowly to room temperature with stirring for 14 hours. The precipitate was collected by vacuum filtration and air-dried. The crude product was recrystallized from 8:2 CH₂Cl₂/MeOH to provide 108 mg of yellow solid. The filtrate was concentrated to a yellow solid by rotavapor. Chromatography with 9.5:0.2 CH₂Cl₂/MeOH afforded 18.6 mg. The total yield of yellow solid was 126.6 mg (100%): R_{f}=0.48 (9.5:0.2 CH₂Cl₂/MeOH); ¹H NMR (400 MHz, CDCl₃) δ 3.95 (s, 3H), 7.34 (ddd, J=3.3, 3.8, 3.3 Hz, 1H), 7.50 (t, J=4.1 Hz, 2H), 7.64 (m, 1H), 8.86 (d, J=11.3 Hz, 1H); ¹³C NMR (200 MHz, CDCl₃) δ 51.6, 96.3, 119.2, 130.6, 134.0, 136.1, 139.6, 154.6, 158.6, 163.8, 165.1; MS (FAB) calcd (M+H)⁺ (C₁₁H₈O₄) 205.18, found 205.11; (MALDI-TOF) found 205.24; Anal. Calcd for C₁₁H₈O₄: C, 64.71; H, 3.95. Found: C, 64.26; H, 3.89; UV (MeCN) λₘₐₓ=400 nm.

### Example 3

### 1-(methoxycarbonyl)-2-methylazulene (3)

A yellow suspension of lactone 2 (660 mg, 3.23 mmol) and 2,2-dimethoxy propane (2 mL, 16.20 mmol) in anhydrous toluene (3 mL) in an ACE pressure sealed tube was heated slowly from room temperature to 200° C. in a period of 2 hours. The temperature was kept constant for a period of 24 hours. The brownish-red solution was introduced directly onto a silica gel flash column using 1:1 CH₂Cl₂/hexanes to afford the brownish-red viscous liquid 646.5 mg (100%): R_{f}=0.63 (CH₂Cl₂); ¹H NMR (400 MHz, CDCl₃) δ 2.83 (s, 3H), 3.98 (s, 3H), 7.13 (s, 1H), 7.39 (t, J=11.1 Hz, 1H), 7.50 (t, J=11.1 Hz, 1M), 7.69 (t, J=11.1 Hz, 1H), 8.28 (d, J=10.6 Hz, 1H), 9.48 (d, J=10.6 Hz, 1H), ¹³C NMR (200 MHz, CDCl₃) δ 18.1, 50.8, 86.2, 115.1, 120.2, 126.8, 127.7, 135.8, 137.2, 142.1, 143.2, 154.1, 166.6; LRMS (EI) calcd M⁺ (Cl₃H₁₂O₂) 200.2366, found 200.0833; LRMS (MALDI-TOF) found 201.22; UV-vis (MeCN) λₘₐₓ=524 nm.

### Example 4

### 2-methylazulene (6)

A red viscous solution of compound 3 (200 mg, 0.99 mmol) in anhydrous H₃PO₄ (3 mL) was heated at 100° C. for 5 minutes. The reaction mixture was poured onto ice-cold water (25 mL) after cooling to room temperature. The resulting purple solution was extracted with EtOAc (3×10 mL). The combined organic extracts were washed with water (3×10 mL), dried over MgSO₄, filtered, and concentrated to a deep purple oil. Chromatography with 95:5 hexanes/Et₂O afforded 139.9 mg (99.3%) of a deep purple oil: R_{f}=0.8 (95:5 hexane/Et₂O); ¹H NMR (400 MHz, CDCl₃) δ 2.67 (s, 3H), 7.14 (t, J=9.8 Hz, 2H), 7.18 (s, 2H), 7.47 (t, J=9.6 Hz, 1H), 8.16 (d, J=9.4 Hz, 2H); ¹³C NMR (200 MHz, CDCl₃) δ 16.7, 118.3, 123.0, 134.1, 135.3, 140.7, 150.3; HRMS (CI) calcd M⁺ (C₁₁H₁₀) 142.2000, found 142.0781

### Example 5

### 2,4-dimethyl azulene (9)

This procedure is adapted from Chen et al (Chen, S. L., et al. J. Org. Chem. 1998, 423-33). A 1.4M solution of MeLi (3.20 mL, 4.39 mmol) was added to a flame-dried flask containing a solution of 2-methyl azulene 6 (520 mg, 3.66 mmol) in anhydrous ether at room temperature. The mixture was subsequently heated to reflux overnight. The resulting suspension was treated with MeOH (2 mL) at −70° C. then with 2N HCl (10 mL) at room temperature. The organic layer was separated, and the aqueous solution was extracted with ether (3×20 mL). The combined organic solution was dried over MgSO₄, removed in vacuo. The brown residue was redissolved in benzene (10 mL), then p-chloranil (899 mg, 3.66 mmol) was added portionally at room temperature. During this time, the solution changed gradually from brown to purple. After 48 h, the reaction was quenched with 1N NaOH (20 mL). The organic layer was washed with water, dried over MgSO₄, filtered, and concentrated to a purple oil. Chromatography with hexane afforded 300 mg (52%) of a deep purple oil: R_{f}=0.4 (hexane); ¹H NMR (200 MHz, CDCl₃) δ 2.65 (s, 3H), 2.85 (s, 3H), 7.02-7.15 (m, 3H), 7.20 (d, J=6.4 Hz, 1H), 7.42 (t, J=9.8 Hz, 1H), 8.17 (d, J=9.5 Hz, 1H); ¹³C NMR (200 MHz, CDCl₃) δ 16.6, 24.3, 116.5, 118.3, 122.0, 126.5, 134.5, 138.1, 140.1, 144.1, 148.4; LRMS (MALDI-TOF) calcd (M+H)⁺ (C₁₂H₁₂) 157.23, found 157.42.

### Example 6

### 4-azulenepropanoic acid, 2-methyl (10)

A 2.5 M solution of n-BuLi (846 μL, 2.12 mmol) was added dropwise to a solution of 2,4-dimethyl azulene 9 (220 mg, 1.41 mmol) and diisopropyl amine (316 μL, 2.26 mmol) in ether (10 mL) at −40° C. then reaction mixture was warmed up to 0° C. for 30 minutes. A solution of bromoacetic acid (195.90 mg, 1.41 mmol) in ether (1 mL) was added dropwise to the reaction mixture after cooling the flask to −40° C. The reaction was allowed to stir overnight as the temperature slowly raised up to room temperature. The reaction mixture was poured onto ice-cold water (30 mL) and the unreacted starting material 12 was extracted with ether (2×10 mL). The aqueous phase was suspended in a mixture of ether (20 mL) and 2M HCl (20 mL). The organic solution was collected, dried over MgSO₄, filtered, and concentrated to a wet purple residue. Chromatography with CH₂Cl₂ afforded 91 mg (30%) of a deep purple oil: R_{f}=0.1 (CH₂Cl₂); LRMS (MALDI-TOF) calcd (M+H)⁺ (C₁₄H₁₄O₂) 215.26, found 215.30 (NMR were not satisfactorily obtained due to internal impurity).

### Example 7

### (NIRQ700, III)

4-azulenepropanoic acid, 2-methyl 10 (83 mg, 0.39 mmol) and squaric acid (22.2 mg, 0.19 mmol) were heated to reflux for 5 h in a solvent mixture containing toluene (20 mL) and n-BuOH (20 mL), accompanied by water removal using Dean-Stark apparatus. The solvents were removed by rotavapor then the residue was redissolved in chloroform. Chromatography with 9:1 CH₂Cl₂/acetone afforded a wet, green sticky plating material (42 mg, 40%). R_{f}=0.34 (9.5:0.5 CHCl₃/CH₃OH); ¹H NMR (200 MHz, CDCl₃, CD₃OD) δ 0.93 (t, J=7.3 Hz, 3H), 1.33 (m, J=7.9 Hz, 2H), 1.61 (q, J=6.4 Hz, 2H), 2.84 (t, J=7.0 Hz, 4H), 3.14 (s, 6H), 3.50 (t, J=7.9 Hz, 4H), 4.11 (t, J=6.7 Hz, 2H), 7.44 (s, 2H), 7.77 (m, 6H), 10.29 (d,d, J=4.27, 4.27, 2H) ¹³C NMR (50 MHz, CDCl₃, CD₃OD) δ 13.74, 19.23, 29.90, 30.83, 33.96, 35.28, 65.18, 125.14, 130.97, 134.62, 139.03, 141.37, 146.65, 148.88, 149.96, 155.47, 155.55, 173.08, 175.05, 182.74, 185.65; HRMS (ES) calcd (M⁺) (C₃₆H₃₅O₆) 563.2433, found 563.2431 UV-vis (ACN) λₘₐₓ=701 nm.

### Example 8

### 1-methyl,4-propanoic,7-isopropyl azulene (13)

This compound was prepared from compound 12 using the same procedure outlined in Example 6. Purification by chromatography with 9.5:0.1 CH₂Cl₂/MeOH) provided 13 as purple crystals (74%): R_{f}=0.5 (9.5:0.5 CH₂Cl₂/MeOH); ¹H NMR (200 MHz, CDCl₃) δ 1.36 (d, J=7.0 Hz, 6H), 2.67 (s, 3H), 2.88 (t, J=4.0 Hz, 2H), 3.12 (m, J=7.0 Hz, 1H), 3.5 (t, J=3.7 Hz, 2H), 7.04 (d, J=10.7 Hz, 1H), 7.29 (d, J=3.7 Hz, 1H), 7.46 (dd, J=12.2 Hz, 1.8 Hz, 1H), 7.65 (d, J=4.0 Hz, 1H), 8.2 (d, J=1.83 Hz, 1H); ¹³C NMR (50 MHz, CDCl₃) δ 12.87, 24.70, 32.96, 35.20, 38.27, 112.10, 124.14, 125.63, 133.53, 135.26, 136.62, 140.44, 146.01, 178.60 LRMS (MALDI-TOF) calcd M⁺ (C₁₇H₂₀O₂) 256.34, found 256.51.

### Example 9

### NIRQ750 (II)

This compound was prepared from compound 13 using the procedure outlined in Example 7 and was obtained in 53% yield. Data for (II): R_{f}=0.34 (9:1 CHCl₃/acetone); ¹H NMR (200 MHz, CDCl₃) δ 0.75 (t, J=7.9 Hz, 3H), 1.19 (m, 2H), 1.39 (d, J=7.0 Hz, 14H), 2.55 (s, 6H), 2.63 (t, J=7.3 Hz, 2H), 3.11 (m, 4H), 3.91 (t, J=6.7 Hz, 2H), 4.08 (t, J=7.9 Hz, 2H), 4.18 (t, J=7.6 Hz, 2H), 7.63 (m, 4H), 8.13 (s, 2H), 8.80 (s, 1H), 9.03 (s, 1H);); ¹³C NMR (50 MHz, CDCl₃) δ 13.1, 13.6, 19.0, 24.2, 30.5, 34.0, 34.8, 35.8, 36.4, 38.4, 64.6, 121.1, 131.5, 134.4, 138.5, 139.9, 141.8, 148.2, 151.3, 152.6, 154.6, 172.9, 173.7, 178.8, 183.9; HRMS (ES) calcd (M⁺) (C₄₂H₄₇O₆) 647.3372, found 647.3364, UV-vis (ACN) λₘₐₓ=755 nm.

Synthesis of Near-Infrared Fluorescence Probes

### Example 10

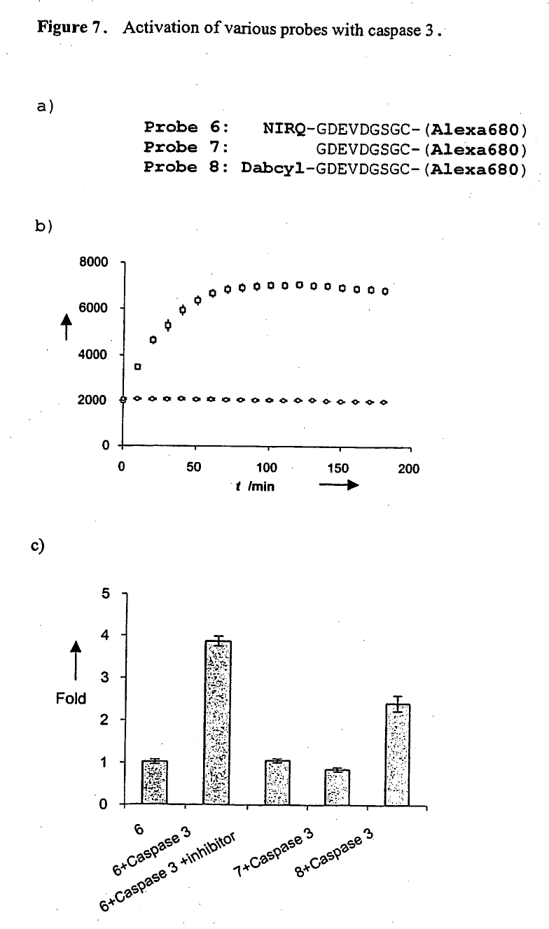

To test the utility of the NIR quencher we designed an activatable NIR fluorescent caspase-3 substrate. The NIRQ750 activated with HBTU/HOBt was directly anchored to the N-terminus of a caspase-3 cleavable peptide substrate GDEVDGSGC (SEQ ID NO: 2). The labeled peptide was then cleaved off the solid support and purified by reverse phase HPLC. The NIRQ retained its optical property through harsh acid treatment during peptide cleavage and deprotection. Thereafter, a near-infrared fluorochrome, Alexa-680 maleimide, Ex=679 nm and Em=702 nm, was reacted with the cystein residue of the opposite end of the peptide chain. Alexa-680 was chosen as the fluorescence donor because of its overlapping emission spectrum with NIRQ (FIG. 2). Two other Alexa-680 containing probes, with no quencher or Dabcyl, were also prepared as controls (FIG. 7A).

Probe Activation

### Example 11

The HPLC purified NIRF probes were tested against caspase-3 with or without an inhibitor of the enzyme in pH7.2 buffer (20 mM PIPES, 100 mM NaCl, 10 mM DTT, 1 mM EDTA, 0.1% CHAPS, 10% sucrose). Within 180 minutes, the fluorescence signal of NIRQ750-probe (probe 6, FIG. 7A) had increased about 4-fold while no activation was observed when the caspase-3 specific inhibitor was present (FIG. 7B). Since Dabcyl is not an ideal quencher in this optical window, the fluorescence signal of this probe (Probe 8, FIG. 7A) was only increased by 2.4-fold (FIG. 7C). As expected, the fluorescence signal of both negative controls, (probe 7, FIG. 7A) with enzyme and probe 6 without enzyme, were remained unchanged. The result indicates that these probes are stable in DTT containing buffer. An initial study using Stern-Volmer plot suggests that fluorescence quenching is caused by resonance energy transfer (J. R. Lakowicz, Principles of Fluorescence Spectroscopy, 2^{nd} ed., Kluwer, New York, 1999).

In Vivo Imaging

The techniques descibed in “In vivo imaging of tumors with protease-activated near-infrared fluorescent probes” Weissleder R, Tung C H, Mahmood U, Bogdanov A, Jr., Nature Biotechours. 1999; 17, 375-378, and references therein are used and are hereby incorporated by reference.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An intramolecularly-quenched fluorescence probe comprising a spacer, a near-infrared bisazulene dimer quencher, and a near-infrared fluorochrome, wherein said fluorochrome and said quencher are covalently linked to the spacer at fluorescence-quenching permissible positions and are separated by a target-specific activation site.

2. The probe of claim 1, further comprising a polymer backbone linked to the spacer.

3. The probe of claim 2, wherein the polymer backbone comprises a polysaccharide, a nucleic acid, polypeptide or a synthetic polymer.

4. The probe of claim 3, wherein the polymer backbone further includes at least one protective chain covalently linked to the backbone.

5. The probe of claim 4, wherein the proctective chain comprises polyethylene glycol, methoxypolyethylene glycol, methoxypolypropylene glycol, copolymers of polyethylene glycol and methoxypolypropylene glycol, dextran, or polylactic-polyglycolic acid.

6. The probe of claim 1, wherein the spacer comprises a peptide, an oligonucleotide, or a synthetic cleavable moiety.

7. The probe of claim 1, wherein the fluorochrome comprises a Cy5.5, Cy5, Cy 7, Alexa Fluoro 680, Alexa Fluoro 750, IRD41, IRD700, NIR-1, LaJolla Blue, indocyanine green (ICG), indotricarbocyanine (ITC), a chelated lanthanide compound or other near-infrared fluorochromes.

8. The probe of claim 1, further comprising a targeting moiety linked to the spacer.

9. The probe of claim 8, wherein the targeting moiety comprises an antibody, an antigen-binding antibody fragment, a receptor-binding polypeptide, and a receptor-binding polysaccharide.

10. An azulene dimer-quencher compound having the formula (I): 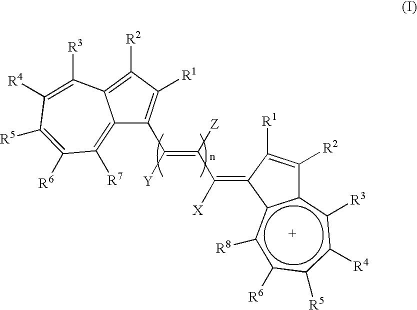
wherein:
R¹, R², R³, R⁴, R⁵, and R⁶ are independently selected from hydrogen, C₁-C₁₂ alkyl, cycloalkyl, heterocyclyl, aryl, heteroaryl, halo, hydroxy, nitro, sulfate, phosphate haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups;
n is independently 1, 2, 3 or 4;
X is independently hydrogen, carbonyl, CH═CH, or when taken together with Y may form a ring;
Y is independently hydrogen or a chemical bond when taken together with X to form a ring;
Z is independently hydrogen or an oxygen radical;
R⁷ and R⁸ are CH₂(CH₂)ₘG;
m is independently 0-12;
G is independently C(O)OH, C(O)OR⁹, C(O)O—NR¹⁰R¹¹, or O—S(O)₂R¹² with the proviso that only one G of R⁷ or R⁸ may be C(O)OR⁹;
R⁹ is C₁-C₁₂ alkyl;
R¹⁰ and R¹¹ taken together form di-oxoheterocyclyl or heteroaryl; and
R¹² is aryl optionally substituted with halo or nitro.

11. The compound of claim 10, wherein R⁸ is CO₂H.

12. The compound of claim 10, wherein R⁸ is:

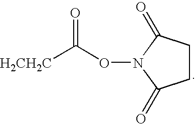

13. The compound of claim 10, wherein R⁸ is:

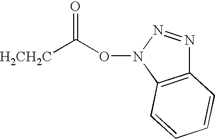

14. The compound of claim 10, wherein n is 1, Z is O⁻, and X and Y combine to form:

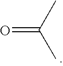

15. An azulene dimer-quenched, near-infrared fluorescent probe having a formula (I): 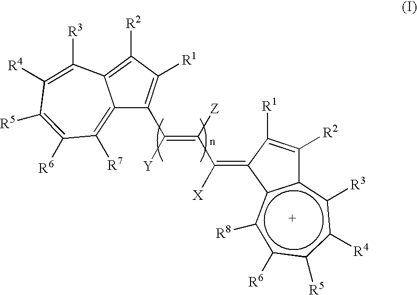
wherein:
R¹, R², R³, R⁴, R⁵, and R⁶ are independently selected from hydrogen, C₁-C₁₂ alkyl, cycloalkyl, heterocyclyl, aryl, heteroaryl, halo, hydroxy, nitro, sulfate, phosphate, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups;
n is independently 1, 2, 3 or 4;
X is independently hydrogen, carbonyl, CH═CH, or when taken together with Y may form a ring;
Y is independently hydrogen or a chemical bond when taken together with X to form a ring;
Z is independently hydrogen or an oxygen radical;
R⁷ and R⁸ are CH₂(CH₂)ₘG;
m is independently [0-12];
G is independently C(O)OR⁹, C(O)NHK, or SL with the proviso that only one G in R⁷ and R⁸ may be C(O)OR⁹;
R⁹ is C₁-C₁₂ alkyl;
K is an N-terminal amino acid of an amino acid sequence containing a fluoroscence activation site within the sequence and a near-infrared fluorochrome covalently bonded to the amino acid sequence directly or through a spacer; and
L is a cysteine residue of an amino acid sequence containing a fluoroscence activation site within the sequence and a near-infrared fluorochrome covalently bonded to the amino acid sequence directly or through a spacer.

16. The probe of claim 15, wherein K is glycine, the amino acid sequence is Gly-Asp-Glu-Val-Asp-Gly-Ser-Gly-Cys-NH₂ (SEQ ID NO: 1) and the fluorochrome is Alexa-680 C₂ maleimide.

17. An in vivo method of detecting a target in a subject, the method comprising:
(a) administering to a subject a probe of claim 1, that accumulates preferentially in the target;
(b) allowing time for (1) the probe to accumulate preferentially in the target, and (2) enzymes in the target to activate the probe by enzymatic cleavage at a fluorescence activation site;
(c) illuminating the target with near-infrared light of a wavelength absorbable by the fluorochromes; and
(d) detecting fluorescence emitted by the fluorochromes.

18. The method of claim 17, wherein the fluorescence emitted in (d) is used to form an image of the target.

19. The method of claim 17, wherein the subject is a mammal.

20. The method of claim 19, wherein the subject is a human.

21. The method of claim 17, wherein the target is a tumor.

22. The method of claim 17, wherein (a), (b), (c), and (d) are repeated over time.

23. The method of claim 17, wherein the illuminating step and the detecting step are performed endoscopically.

24. The method of claim 17, wherein (d) is performed using a suitable light detection or image recording component consisting of a charged coupled device (CCD) system or photographic film.

25. The method of claim 17, wherein the presence, absence, or level of probe activation is indicative of a disease state.

26. The method of claim 25, wherein the disease state is cancer.

27. An in vivo method of detecting or evaluating an arthritic area in a joint of a subject, the method comprising:
(a) administering to a subject a probe of claim 1, that accumulates preferentially in an arthritic area;
(b) allowing time for (1) the probe to accumulate preferentially in the arthritic area, and (2) enzymes in the arthritic area to activate the probe by enzymatic cleavage at a fluorescence activation site;
(c) illuminating the arthritic area with near-infrared light of a wavelength absorbable by the fluorochromes; and
(d) detecting fluorescence emitted by the fluorochromes.

28. The method of claim 27, wherein the illuminating step and the detecting step are performed endoscopically.

29. An in vivo method of selectively detecting two different cell or tissue types simultaneously, the method comprising:
(a) administering to a subject two different probes of claim 1, each of which accumulates preferentially in a target tissue, wherein each of the two probes comprises a fluorochrome whose fluorescence wavelength is distinguishable from that of the other flurorochrome, and each of the two spacers comprises a different activation site:
(b) allowing time for (1) the probes to accumulate preferentially in the target tissue, and (2) enzymes in the target tissue to activate the probes by enzymatic cleavage at a fluorescence activation site, if the target tissue is present;
(c) illuminating the target tissue with near-infrared light of a wavelength absorbable by the fluorochromes; and
(d) separately detecting fluorescence emitted by the two fluorochromes.

30. The method of claim 29, wherein the fluorescence emitted by the two different fluorochromes in (d) is used to form an image of the two different cell or tissue types simultaneously.


SEQUENCE LISTING (2 sequences)

SEQ ID NO: 1 - protein, 9 aa, Artificial Sequence
  Synthetically generated peptide
GDEVDGSGC

SEQ ID NO: 2 - protein, 9 aa, Artificial Sequence
  Synthetically generated peptide
GDEVDGSGC